(19) United States
(12) Patent Application Publication
(10) Pub. No.: US 2025/0176970 A1
(43) Pub. Date: Jun. 5, 2025

(54) Improved Surgical Clamping Instrument with Emergency Release

(71) Applicant: USB Medical, Ltd., Hatboro, PA (US)

(72) Inventors: John J. Sinisi, Warminster, PA (US); Jesse Sinisi, Warminster, PA (US); Stephen T. Epstein, Newtown, PA (US); Mattie Glauber, Milano (IT)

(21) Appl. No.: 18/703,295

(22) PCT Filed: Oct. 19, 2022
(86) PCT No.: PCT/US2022/047193

§ 371 Date: Apr. 19, 2024

Related U.S. Application Data
(63) Continuation-in-part of application No. 17/506,611, filed on Oct. 20, 2021, now abandoned.
(60) Provisional application No. 63/359,811, filed on Jul. 9, 2022.

(51) Int. Cl. A61B 17/128; A61B 17/00; A61B 17/29

(52) U.S. Cl. CPC A61B 17/1285; A61B 17/29; A61B 2017/00367; A61B 2017/2929; A61B 2017/2931; A61B 2017/2932; A61B 2017/2946; A61B 2017/2947

(57) ABSTRACT

A clamping system for use during a surgical procedure and the clamping head used in such a system. The clamping system includes a clamp head, forceps, and a mechanical actuator. The clamp head has a housing, a first jaw and a second jaw. The first jaw is attached to the housing with a hinged connection. A removable retention clip maintains the hinged connection. A ring clamp is provided on the forceps that opens and closes with the forceps. The ring clamp receives and retains the housing of the clamp head. The ring clamp can be closed around a neck of the housing across a wide range of approach angles. A mechanical actuator is attached to the forceps. The mechanical actuator has an elongated shaft that aligns with the clamp head when the ring clamp is closed around the tubular neck of the clamp head.

### TECHNICAL FIELD OF THE INVENTION

In general, the present invention relates to surgical clamping instruments. More particularly, the present invention relates to surgical clamping instruments that have clamp heads that can detach from a handle inside the body. In this manner, a clamp head can be left within an incision while its handle is removed to make room for other surgical instruments.

### BACKGROUND ART

There are many surgical procedures where a clamp is used deep within an incision. Traditional clamps have handles that are used to manipulate the clamp into position. The handles are also used to selectively open and close the clamp. The problem with such traditional clamps is that the handle of the clamp creates a physical barrier within the incision. The handle of the clamp can prevent a surgeon from viewing and/or accessing tissue of interest. The presence of the clamp handle also limits the room in which a surgeon can insert and manipulate other surgical instruments.

In the prior art, one solution to the clamp crowding problem involves utilizing a clamp with a clamp head that detaches from its handle. In this manner, the clamp head can be set in place using the handle, then the handle can be removed. The clamp head remains in place and the handle is no longer an obstruction. One such prior art clamping system is commonly referred to as a Glauber clamp and is exemplified by U.S. Pat. No. 8,409,229 to Wiedenbein.

There are certain disadvantages associated with prior art clamping systems, such as the Glauber clamp. One disadvantage is that once the clamp head is detached from the handle inside the body, it is often very difficult to reattach the handle to the clamp head inside the body. The clamp head and the handle must be specifically oriented before the clamp head and the handle can interconnect. Since the clamp head can, and often does, change orientations within the body, the clamp head rarely aligns with the handle when the handle is reinserted through an incision. The clamp head must then be manipulated into a better orientation using other surgical instruments. However, the presence of the other surgical instruments limits the room available to maneuver the clamp handle. Thus, the problem becomes self-complicating.

Another disadvantage of prior art Glauber-style clamping systems is that the clamps have large connection hubs. The connection hubs present large obstructions deep within surgical incisions. However, the connection hubs are required to receive the detachable handles and to transfer mechanical action from the handle to the clamp head to open and close the clamp head. If this connection cannot be made, or if the clamp head jams and cannot be opened using the handle, the clamp must be surgically removed by other means. This adds significant complications to the surgical procedure.

A need therefore exists for an improved surgical clamping system that has a clamp head that detaches from a handle, wherein corresponding parts can attach throughout a wide range of orientations. A need also exists for a detachable clamp head with a less bulky attachment hub and a detachable clamp that can be easily opened and removed. Furthermore, a need exists for an improved clamp head that can be used during a minimally invasive surgical procedure, where the clamp head contains an emergency release that can be activated by other instruments from within the body. These needs are met by the present invention as described below.

### DISCLOSURE OF THE INVENTION

The present invention is a clamping system for use during a surgical procedure and the clamping head used in such a system. The clamping system includes a clamp head, forceps, and a mechanical actuator. The clamp head has a housing, where a first jaw and a second jaw extend from the housing. The housing has a tubular neck and a drive shaft that extends through the tubular neck. The first jaw is attached to the housing with a hinged connection. The first jaw can be moved relative to the second jaw by selectively rotating the drive shaft. A retention clip maintains the hinged connection between the housing and the first jaw. The first jaw is readily detachable from the housing when the retention clip is removed. Accordingly, the clamp head can be opened in an emergency by removing the retention clip using a secondary instrument.

The forceps can be manually opened and closed. A ring clamp is provided on the forceps that opens and closes with the forceps. The ring clamp is sized to receive and retain the tubular neck on the housing of the clamp head. The ring clamp can be closed around the neck across a wide range of approach angles. This is achieved, in part, by a unique side bevel on both the neck and the forceps that enable an off-center approach with a self-centering connection.

A mechanical actuator is attached to the forceps. The mechanical actuator has an elongated shaft that aligns with the drive shaft of the clamp head when the ring clamp is closed around the tubular neck of the clamp head. The alignment enables the mechanical actuator to engage the drive shaft and turn the drive shaft when the mechanical actuator is manually turned.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 is a perspective view of an exemplary embodiment of a clamp assembly with the clamp head attached to forceps;

FIG. 2 is a perspective view of the embodiment of FIG. 1 with the clamp head separated from the forceps;

FIG. 3 is an exploded view of the forceps of the exemplary embodiment;

FIG. 4 is an enlarged perspective view of the clamp head shown with a fragment of the forceps; and

FIG. 5 is an exploded view of the exemplary embodiment of the clamp head shown in FIG. 4.

### DETAILED DESCRIPTION OF BEST MODE FOR CARRYING OUT THE INVENTION

Although the present invention clamping system can be embodied in many ways, only one exemplary embodiment is illustrated. The exemplary embodiment is shown for the purposes of explanation and description. The exemplary embodiment is selected in order to set forth one of the best modes contemplated for the invention. The illustrated embodiment, however, is merely exemplary and should not be considered a limitation when interpreting the scope of the appended claims.

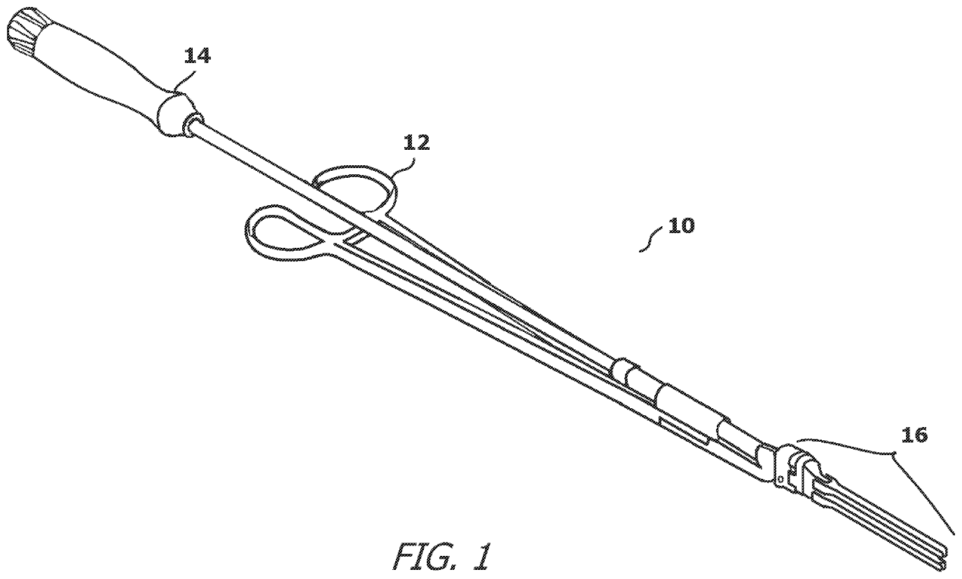

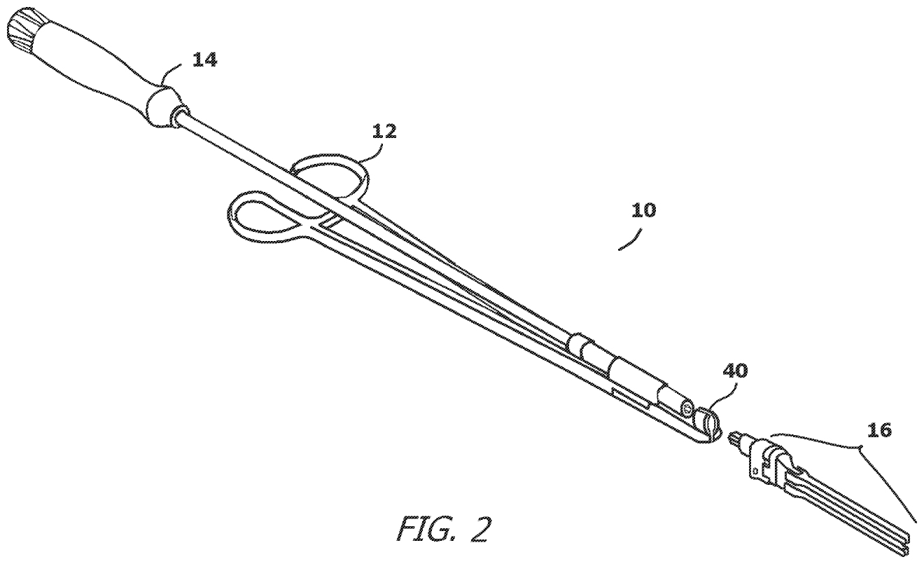

Referring to FIG. 1 and FIG. 2, a clamping system 10 is shown. The clamping system 10 includes specialized forceps 12, a mechanical actuator 14, and a clamp head 16. The specialized forceps 12 support the mechanical actuator 14. The specialized forceps 12 are also designed to selectively engage and release the clamp head 16. As such, the specialized forceps 12 are used to both insert the clamp head 16 into an incision and remove the clamp head 16 from an incision. The mechanical actuator 14 is used to selectively open and close the clamp head 16 when the clamp head 16 is engaged with the specialized forceps 12.

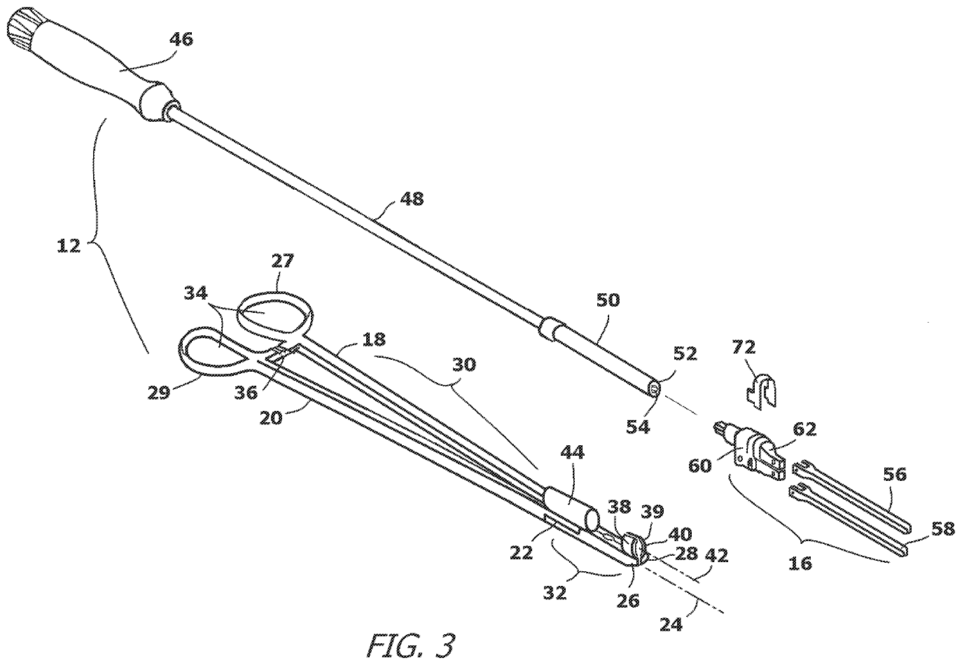

Referring to FIG. 3 in conjunction with FIG. 1 and FIG. 2, it can be seen that the specialized forceps 12 have two shanks 18, 20 that are joined together at a common scissor joint 22. The specialized forceps 12 are symmetrically disposed about a center line 24 that bisects the scissor joint 22. The first shank 18 has a first end 26 and an opposite second end 27. Likewise, the second shank 20 has a first end 28 and an opposite second end 29. The scissor joint 22 divides the specialized forceps 12 between a handle section 30 and a jaw section 32. The handle section 30 extends from the scissor joint 22 to the first ends 26, 28 of the shanks 18, 20. The first ends 26, 28 of the shanks 18, 20 terminate with finger loops 34. This allows the handle section 30 of the specialized forceps 12 to be gripped and manipulated in a traditional manner. The shanks 18, 20 can also include ratcheted extensions 36 that enable the two shanks 18, 20 to be selectively locked together at different angles.

The jaw section 32 of the specialized forceps 12 extends from the scissor joint 22 to the second ends 27, 29 of the two shanks 18, 20. At the second ends 27, 29 of the shanks 18, 20, each of the shanks 18, 20 terminates with a half ring 38, 39. Each half ring 38, 39 extends away from the shanks 18, 20 in a direction perpendicular to the bisecting centerline 24. When the jaw section 32 is closed and the shanks 18, 20 abut, the two half rings 38, 39 align to form a two-part ring clamp 40 with an internal radius. The two-part ring clamp 40 has a central axis 42 that is parallel to the bisecting centerline 24.

A guide tube 44 is mounted to the first shank 18 near the scissor joint 22. When the jaw section 32 is closed and the shanks 18, 20 abut, the guide tube 44 concentrically aligns with the two-part ring clamp 40, wherein both the guide tube 44 and the two-part ring clamp 40 share the same central axis 42.

The mechanical actuator 14 includes a handle 46. An elongated shaft 48 extends from the handle 46, wherein the elongated shaft 48 terminates at an engagement head 50. The engagement head 50 is cylindrical in shape and terminates at a distal end 52. A receptacle 54 is formed in the distal end 52 of the engagement head 50 that has a keyed shape, such as a hex shape or a spline shape. The purpose for the receptacle 54 is later explained. The engagement head 50 passes through, and is supported by, the guide tube 44 on the specialized forceps 12. The result is that the elongated shaft 48 is aligned with the central axis 42 of the guide tube 44. The mechanical actuator 14 is attached to the specialized forceps 12 in a manner where the elongated shaft 48 is free to rotate about the central axis 42. The elongated shaft 48 can be selectively rotated by manually turning the handle 46.

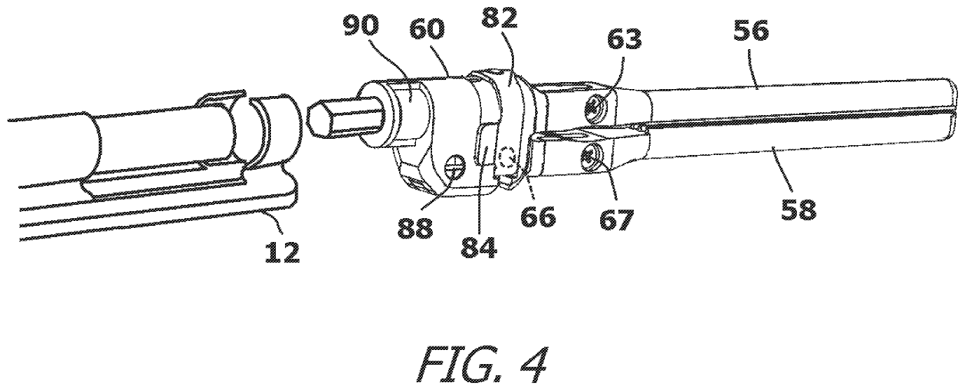

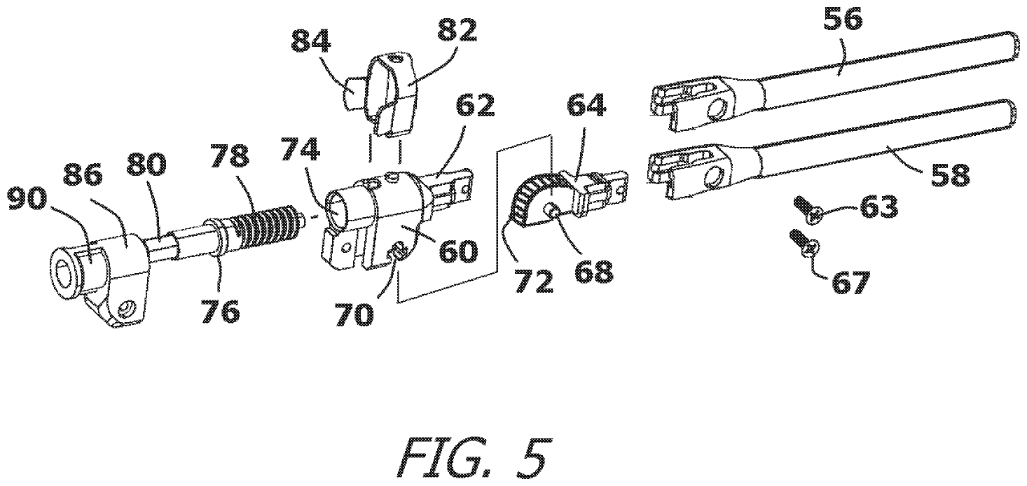

Referring to FIG. 4 and FIG. 5 in conjunction with FIG. 3, it can be seen that clamp head 16 has two jaws 56, 58. The jaws 56, 58 include a stationary jaw 56 and an articulating jaw 58. When the clamp head 16 is closed, the stationary jaw 56 and the articulating jaw 58 abut along their lengths. When the clamp head 16 is open, the articulating jaw 58 diverges away from the stationary jaw 56 at an angle of inclination. As is later explained, the stationary jaw 56 and the articulating jaw 58 are both selectively detachable from the clamp head 16. In this manner, jaws of different sizes and shapes can be used on the clamp head 16. In the shown embodiment, the jaws 56, 58 are straight. However, it should be understood that the jaws 56, 58 can be curved, wherein the shape of the jaws 56, 58 are selected to match the needs of a surgical procedure.

The clamp head 16 has a primary housing 60. A fixed mount 62 extends from the primary housing 60. The stationary jaw 56 selectively connects to the fixed mount 62 with a first mechanical fastener 63. As such, it will be understood that jaws of different shapes and sizes can be attached to the fixed mount 62 using the first mechanical fastener 23.

A pinioned linkage 64 is provided. The pinioned linkage 64 is used to connect the articulating jaw 58 to the primary housing 60. The pinioned linkage 64 connects to the primary housing 60 at a hinged connection 66. The pinioned linkage 64 connects to the articulating jaw 68 with a second mechanical fastener 67. The hinged connection 66 is made by providing hinge pins 68 on the pinioned linkage 64 that connect into slots 70 in the primary housing 60. The pinioned linkage 64 terminates at one end with an arcuate section of a pinon gear 72 that is radially disposed about the axis of the hinge pins 68.

Since the stationary jaw 56 and the articulating jaw 58 are attached using mechanical fasteners 63, 67, it will be understood that jaws 56, 56 can be readily changed to meet the needs of a surgical procedure. Jaws that are curved, angled, or looped can be utilized as part of the clamp head 16. It will therefore be understood that the straight jaws 56, 58 illustrated are merely exemplary and are meant to represent jaws of any shape and size.

A conduit 74 extends into the primary housing 60. When the pinioned linkage 64 is attached to the primary housing 60, the conduit 74 intersects the pinon gear 72 of the pinioned linkage 64 at a tangent. A worm gear structure 76 is provided. The worm gear structure 76 has a threaded section 78 that advances into the conduit 74 of the primary housing 60 and intermeshes with the pinion gear 72 of the pinioned linkage 64. The worm gear structure 76 is turned by a hex drive shaft 80 that extends out of the primary housing 60. When the hex drive shaft 80 rotates, the rotational action is transferred to the threaded section 78 of the worm gear structure 76. As the treaded section 78 rotates, it causes the pinion gear 72 and the attached pinioned linkage 64 to rotate. The pinioned linkage 64, therefore, rotates relative to the primary housing 60 about the hinged connection 66. The pinioned linkage 64 is attached to the articulating jaw 58. Accordingly, as the pinioned linkage 64 moves, the articulating jaw 58 moves in relation to the stationary jaw 56.

The hinge pins 68 on the pinioned linkage 64 rotate in the slots 70 that are formed in the primary housing 60. The hinge pins 68 are capable of being pulled out of the slots 70 upon the application of a small pulling force. To prevent the hinge pins 68 from inadvertently pulling out of the slots 70, a retention clip 82 is provided. The retention clip 82 extends around the primary housing 60 and engages the hinge pins 68 on both sides of the primary housing 60. When the retention clip 82 is in place, the hinge pins 68 are incapable of separating out of the slots 70.

The retention clip 82 has flared tabs 84 that extend slightly from the primary structure of the retention clip 82. The flared tabs 84 make it possible to engage the retention clip 82 and lever the retention clip 82 away from the primary housing 60. The flared tabs 84 make it possible to remove the retention clip 82 while inside the body using a different surgical instrument, such as another clamp. When the retention clip 82 is disconnected, the hinge pins 68 can be pulled out of the slots 70 on the primary housing 60. This enables the pinioned linkage 64 to disengage from the primary housing 60. Since the articulating jaw 58 is attached to the pinioned linkage 64, the articulating jaw 58 can move away from the stationary jaw 56. In this manner, should the clamp head 16 ever fail to open in the body, the clamp head 16 can be rapidly opened with another surgical instrument without having to create any new surgical incision.

A collar 86 attaches to the primary housing 60. The collar 86 is connected to the primary housing 60 using a mechanical fastener 88. The collar 86 has a tubular neck 90 that surrounds part of the worm gear structure 76. It is the tubular neck 90 of the collar 86 that is engaged by the specialized forceps 12.

Referring to all figures, it will be understood that to use the clamping system 10, the clamp head 16 is attached to the specialized forceps 12 and is advanced into an incision. The clamp head 16 is attached to the specialized forceps 12 by closing the jaw section 32 about the tubular neck 90 on the clamp head 16. More particularly, as the jaw section 32 is closed, the two-part ring clamp 40 closes over the tubular neck 90 on the clamp head 16. The engagement of the two-part ring clamp 40 around the tubular neck 90 aligns the tubular neck 90 and the hex drive shaft 80 of the worm gear structure 76 with the elongated shaft 48 of the mechanical actuator 14. That is, the central axis 42 of the elongated shaft 48 and engagement head 50 is aligned with the worm gear structure 76. Since the two-part ring clamp 40 is circular and the tubular neck 90 is circular, the two-part ring clamp 40 can engage the tubular neck 90 on the clamp head 16 from most any direction. Once the two-part ring clamp 40 is closed around the tubular neck 90, the two-part ring clamp 40 will automatically align the central axis 42 of the elongated shaft 48 with the hex drive shaft 80. This automatic alignment is of great benefit to a surgeon who need only grab the tubular neck 90 on the clamp head 16 with the two-part ring clamp 40 to achieve proper alignment.

Once the elongated shaft 48 and the engagement head 50 are aligned with the hex drive shaft 80, then the handle 46 can be manually manipulated to advance the engagement head 50 toward the hex drive shaft 80. The keyed receptacle 54 on the engagement head 50 receives the hex drive shaft 80. This creates a mechanical interconnection between the mechanical actuator 14 and the hex drive shaft 80. As a consequence, when the handle 46 of the mechanical actuator 14 is manually turned, the hex drive shaft 80 in the clamp head 16 will turn. As the hex drive shaft 80 turns, the worm gear structure 76 turns. The worm gear structure 76 turns the pinioned linkage 64. The rotation of the pinioned linkage 64 causes the articulating clamp jaw 58 to move toward or away from the stationary jaw 56. This selectively opens and closes the clamp jaws 56, 58 as directed by the manipulations of the handle 46 by a surgeon.

The clamp jaws 56, 58 on the clamp head 16 can be adjusted as needed. Once the clamp head 16 is properly adjusted, the engagement head 50 can be pulled away. The shanks 18, 20 of the specialized forceps 12 can be spread, whereby the clamp head 16 will detach from the specialized forceps 12. The specialized forceps 12 and the mechanical actuator 14 are removed from the incision, leaving the clamp head 16 within the incision. To retrieve the clamp head 16 from an incision, the process is reversed. The specialized forceps 12 are advanced into the incision. The two-part ring clamp 40 is closed around the tubular neck 90 of the clamp head 16. This automatically aligns the hex drive shaft 80 of the clamp head 16 with the engagement head 50. The mechanical actuator 14 can then be advanced and turned to mechanically open the clamp head 16. The clamp head 16 can then be removed along with the specialized forceps 12 as a unit.

If the clamp head 16 cannot be engaged by the specialized forceps 12, then the retention clip 82 on the clamp head 16 can be engaged and removed. This acts as an emergency release that enables the jaws 56, 58 of the clamp head 16 to separate and open. The separated parts are then individually removed from the surgical site.

It will be understood that the embodiment of the present invention that is illustrated and described is merely exemplary and that a person skilled in the art can make many variations to that embodiment. For instance, the size and shape of the specialized forceps and the clamp head can be altered to meet the needs of a particular surgical procedure. All such embodiments are intended to be included within the scope of the present invention as defined by the claims.

What is claimed is:

1. A clamping system, comprising:
a clamp head having a housing with a tubular neck, a first jaw that extends from said housing, a second jaw that extends from said housing, and a drive shaft that extends into said housing through said tubular neck, wherein said first jaw can be moved relative to said second jaw by rotating said drive shaft;
forceps that open and close, said forceps having a ring clamp thereon that opens and closes with said forceps, and wherein said ring clamp is sized to receive and retain said tubular neck on said clamp head; and
a mechanical actuator supported by said forceps, wherein said mechanical actuator has an elongated shaft that aligns with said drive shaft when said ring clamp on said forceps is closed around said tubular neck of said clamp head.

2. The clamping assembly according to claim 1, wherein said first jaw is selectively detachable from said housing and is held attached to said housing with a removable retention clip.

3. The clamping assembly according to claim 2, wherein said first jaw has hinge pins extending therefrom that are received by said housing, wherein said removable retention clip engages said hinge pins.

4. The clamping assembly according to claim 2, wherein at least one tab extends from said retention clip to provide an accessible point for engaging said retention clip with a secondary surgical instrument.

5. The clamping assembly according to claim 1, wherein said second jaw is stationary relative to said housing, and said first jaw is moved by rotating said drive shaft.

6. The clamping assembly according to claim 1, wherein said elongated shaft of said mechanical actuator extends along a central axis and said mechanical actuator includes a handle for selectively rotating said elongated shaft about said central axis and reciprocally moving said elongated shaft along said central axis.

7. The clamping assembly according to claim 6, wherein said elongated shaft terminates with an engagement head that moves with said elongated shaft, wherein said elongated shaft selectively interconnects with said drive shaft of said clamp head when said ring clamp on said forceps is closed around said tubular neck of said clamp head.

8. The clamping assembly according to claim 1, wherein said first jaw rotates about a pivot pin.

9. The clamping assembly according to claim 8, wherein said first jaw includes a pinion gear that rotates about said pivot pin.

10. The clamping assembly according to claim 9, wherein said clamp head includes a worm gear that is rotated by said drive shaft, wherein said worm gear turns said pinion gear and moves said first jaw.

11. The clamping assembly according to claim 1, wherein said forceps include a first shank and a second shank joined at a scissor joint, wherein said ring clamp includes a first half ring on said first shank and a second half ring on said second shank.

12. A clamp head for forceps, comprising:
a housing;
a stationary jaw affixed to said housing;
an articulating jaw connected to said housing with a hinged connection;
a shaft that extends into said housing and moves said articulating jaw about said hinged connection as said shaft is rotated within said housing;
a retention clip that maintains said hinged connection, wherein said articulating jaw is detachable from said housing when said retention clip is removed.

13. The clamping head according to claim 12, wherein said articulating jaw has hinge pins extending therefrom that are received by said housing, wherein said retention clip engages said hinge pins.

14. The clamp head according to claim 12, wherein at least one tab extends from said retention clip to provide an accessible point for engaging said retention clip with a secondary surgical instrument.

15. The clamp head according to claim 12, wherein said articulating jaw includes a pinion gear that rotates about a pivot pin.

16. The clamp head according to claim 15, wherein said clamp head includes a worm gear that is rotated by said shaft, wherein said worm gear turns said pinion gear and moves said articulating jaw.
